(19) United States
(12) Patent Application Publication
(10) Pub. No.: US 2017/0190638 A1
(43) Pub. Date: Jul. 6, 2017

(54) Ethylbenzene Production with Ethylene from Oxidative Coupling of Methane

(71) Applicant: Sabic Global Technologies, B.V., BERGEN OP ZOOM (NL)

(72) Inventors: Wugeng LIANG, Richmond, TX (US); Vidya Sagar Reddy SARSANI, Pearland, TX (US); David WEST, Bellaire, TX (US); Aghaddin MAMEDOV, Sugar Land, TX (US); James LOWREY, Lake Jackson, TX (US)

(21) Appl. No.: 15/363,268

(22) Filed: Nov. 29, 2016

Related U.S. Application Data
(60) Provisional application No. 62/274,596, filed on Jan. 4, 2016.

(51) Int. Cl. C07C 2/84; C07C 2/70

(52) U.S. Cl. CPC C07C 2/84; C07C 2/70; C07C 2529/06

(57) ABSTRACT

A method for producing ethylbenzene (EB) comprising introducing to an oxidative coupling of methane (OCM) reactor an OCM reactant mixture comprising CH₄ and O₂; allowing the OCM reactant mixture to react via OCM reaction to form an OCM product mixture comprising C₂H₄, C₂H₆, water, CO, CO₂ and unreacted methane; separating the water and optionally CO and/or CO₂ from the OCM product mixture to yield an EB reactant mixture comprising C₂H₄, C₂H₆, unreacted methane, and optionally CO and/or CO₂; (d) introducing benzene and an EB reactant mixture to an EB reactor; allowing benzene to react in a liquid phase with the ethylene of the EB reactant mixture to form EB; recovering from the EB reactor an EB product mixture comprising EB and unreacted benzene, and an unreacted alkanes mixture comprising C₂H₆ and unreacted methane, and optionally CO and/or CO₂; and optionally recycling the unreacted alkanes mixture to the OCM reactor.

### CROSS-REFERENCE TO RELATED APPLICATIONS

The present application is a non-provisional of and claims priority to U.S. Provisional Patent Application No. 62/274,596 filed Jan. 4, 2016 and entitled “Ethylbenzene Production with Ethylene from Oxidative Coupling of Methane,” which application is incorporated by reference herein in its entirety.

### TECHNICAL FIELD

The present disclosure relates to methods of producing aromatic hydrocarbons, more specifically methods of producing ethylbezene with ethylene from oxidative coupling of methane.

### BACKGROUND

Hydrocarbons, and specifically olefins such as ethylene, can be typically used in a wide variety of industrial processes, for example, polymerization, oxidation, halogenation, hydrohalogenation, alkylation, hydration, oligomerization, and hydroformylation. Ethylene can be typically used to produce a wide variety of chemical compounds, such as ethylbenzene (EB), ethylene oxide, ethylene dichloride, and polyethylene.

Currently, for industrial scale applications, ethylene is produced by heating natural gas condensates and petroleum distillates, which include ethane and higher hydrocarbons, and the produced ethylene is separated from a product mixture by using gas separation processes.

Ethylene can also be produced by oxidative coupling of the methane (OCM) as represented by Equations (I) and (II):

2CH₄+O₂→C₂H₄+2H₂OΔH=−67 kcal/mol (I)

2CH₄+1/2O₂→C₂H₆+H₂OΔH=−42 kcal/mol (II)

Oxidative conversion of methane to ethylene is exothermic. Excess heat produced from these reactions (Equations (I) and (II)) can push conversion of methane to carbon monoxide and carbon dioxide rather than the desired C₂ hydrocarbon product (e.g., ethylene):

CH₄+1.5O₂→CO+2H₂OΔH=−124 kcal/mol (III)

CH₄+2O₂→CO₂+2H₂OΔH=−192 kcal/mol (IV)

The excess heat from the reactions in Equations (III) and (IV) further exasperate this situation, thereby substantially reducing the selectivity of ethylene production when compared with carbon monoxide and carbon dioxide production.

When ethylene is produced by OCM, there are various methods for separating ethylene from the products of OCM, but all of them present the issue of high separation cost. Some separation methods use cryogenic separation for ethylene separation from OCM products, and cryogenic separation is an energy intensive process, thus a very costly process. Other separation methods use a solid adsorbent to adsorb ethylene from the OCM products, then desorb ethylene at higher temperatures. Cyclic adsorption and desorption is a complex operation and it requires a large amount of adsorbent, resulting in a very high cost process. In other instances, ethylene can be further reacted to form C4 or higher molecules after OCM; however, this would result in a very complex product distribution, and separating out the useful ones would still result in a high cost process.

EB is a crucial intermediate in the production of styrene, the precursor to polystyrene, a common plastic material. EB can also be used in fuels, and to produce a wide range of products, such as a solvent in inks, rubber adhesives, varnishes, and paints. EB is generally produced from benzene and ethylene. Thus, there is an ongoing need for the development of processes for the production of olefins such as ethylene, and EB.

### BRIEF SUMMARY

Disclosed herein is a method for producing ethylbenzene (EB) comprising (a) introducing a first oxidative coupling of methane (OCM) reactant mixture to a first OCM reactor, wherein the first OCM reactant mixture comprises methane (CH₄) and oxygen (O₂), (b) allowing at least a portion of the first OCM reactant mixture to react via an OCM reaction to form a first OCM product mixture, wherein the first OCM product mixture comprises ethylene (C₂H₄), ethane (C₂H₆), water, carbon monoxide (CO), carbon dioxide (CO₂) and unreacted methane, (c) separating components of the first OCM product mixture, wherein separating components comprises removing at least a portion of the water and optionally at least a portion of the CO and/or CO₂ from the first OCM product mixture to yield a first EB reactant mixture, and wherein the first EB reactant mixture comprises C₂H₄, C₂H₆, unreacted methane, and optionally CO and/or CO₂, (d) introducing benzene and at least a portion of the first EB reactant mixture to a first EB reactor, (e) allowing a portion of the benzene to react with at least a portion of the ethylene of the first EB reactant mixture to form EB, (f) recovering a first EB product mixture and a first unreacted alkanes mixture from the first EB reactor, wherein the first EB product mixture comprises EB and unreacted benzene, and wherein the first unreacted alkanes mixture comprises C₂H₆ and unreacted methane, and optionally CO and/or CO₂, (g) introducing O₂ and at least a portion of the first unreacted alkanes mixture to a second OCM reactor, (h) allowing at least a portion of the O₂ and at least a portion of the first unreacted alkanes mixture to react via an OCM reaction to form a second OCM product mixture, wherein the second OCM product mixture comprises C₂H₄, C₂H₆, water, CO, CO₂ and unreacted methane, and wherein an amount of unreacted methane in the second OCM product mixture is less than an amount of unreacted methane in the first OCM product mixture, (i) separating components of the second OCM product mixture, wherein separating components comprises removing at least a portion of the water and optionally at least a portion of the CO and/or CO₂ from the second OCM product mixture to yield a second EB reactant mixture, wherein the second EB reactant mixture comprises C₂H₄, C₂H₆, unreacted methane, and optionally CO and/or CO₂, and wherein an amount of unreacted methane in the second EB reactant mixture is less than an amount of unreacted methane in the first EB reactant mixture, (j) introducing at least a portion of the first EB product mixture and at least a portion of the second EB reactant mixture to a second EB reactor, (k) allowing a portion of the benzene of the first EB product mixture to react with at least a portion of the ethylene of the second EB reactant mixture to form EB, (1) recovering a second EB product mixture and a second unreacted alkanes mixture from the second EB reactor, wherein the second EB product mixture comprises EB and unreacted benzene, wherein an amount of unreacted benzene in the second EB product mixture is less than an amount of unreacted benzene in the first EB product mixture, wherein the second unreacted alkanes mixture comprises C₂H₆ and unreacted methane, and optionally CO and/or CO₂, and wherein an amount of unreacted methane in the second unreacted alkanes mixture is less than an amount of unreacted methane in the first unreacted alkanes mixture, and (m) optionally recycling at least a portion of the second unreacted alkanes to the first OCM reactor and/or the second OCM reactor.

Also disclosed herein is a method for producing ethylbenzene (EB) comprising (a) introducing an oxidative coupling of methane (OCM) reactant mixture to an OCM reactor, wherein the OCM reactant mixture comprises methane (CH₄) and oxygen (O₂), (b) allowing at least a portion of the OCM reactant mixture to react via an OCM reaction to form an OCM product mixture, wherein the OCM product mixture comprises ethylene (C₂H₄), ethane (C₂H₆), water, carbon monoxide (CO), carbon dioxide (CO₂) and unreacted methane, (c) separating at least a portion of the water and optionally at least a portion of the CO and/or CO₂ from the OCM product mixture to yield an EB reactant mixture, wherein the EB reactant mixture comprises C₂H₄, C₂H₆, unreacted methane, and optionally CO and/or CO₂, (d) introducing benzene and at least a portion of the EB reactant mixture to an EB reactor, wherein the at least a portion of the EB reactant mixture is pressurized prior to introducing to the EB reactor, (e) allowing a portion of the benzene to react in a liquid phase with at least a portion of the ethylene of the EB reactant mixture to form EB, (f) recovering an EB product mixture and an unreacted alkanes mixture from the EB reactor, wherein the EB product mixture comprises EB and unreacted benzene, and wherein the unreacted alkanes mixture comprises C₂H₆ and unreacted methane, and optionally CO and/or CO₂, and (g) optionally recycling at least a portion of the unreacted alkanes mixture to the OCM reactor.

Further disclosed herein is a method for producing an ethylene derivative (ED) comprising (a) introducing an oxidative coupling of methane (OCM) reactant mixture to an OCM reactor, wherein the OCM reactant mixture comprises methane (CH₄) and oxygen (O₂), (b) allowing at least a portion of the OCM reactant mixture to react via an OCM reaction to form an OCM product mixture, wherein the OCM product mixture comprises ethylene (C₂H₄), ethane (C₂H₆), water, carbon monoxide (CO), carbon dioxide (CO₂) and unreacted methane, (c) separating at least a portion of the water and optionally at least a portion of the CO and/or CO₂ from the OCM product mixture to yield an ED reactant mixture, wherein the ED reactant mixture comprises C₂H₄, C₂H₆, unreacted methane, and optionally CO and/or CO₂, (d) introducing at least a portion of the ED reactant mixture to an ED reactor, (e) allowing at least a portion of the ethylene of the ED reactant mixture to react and form the ED, and (f) recovering an ED product mixture from the ED reactor, wherein the ED product mixture comprises the ED.

Further disclosed herein is a method for producing ethylbenzene (EB) comprising (a) introducing an oxidative coupling of methane (OCM) reactant mixture to an OCM reactor, wherein the OCM reactant mixture comprises methane (CH₄) and oxygen (O₂), (b) allowing at least a portion of the OCM reactant mixture to react via an OCM reaction to form an OCM product mixture, wherein the OCM product mixture comprises ethylene (C₂H₄), ethane (C₂H₆), water, carbon monoxide (CO), carbon dioxide (CO₂) and unreacted methane, (c) separating components of the OCM product mixture, wherein separating components comprises removing at least a portion of the water and optionally at least a portion of the CO and/or CO₂ from the OCM product mixture to yield an EB reactant mixture, wherein the EB reactant mixture comprises C₂H₄, C₂H₆, unreacted methane, and optionally CO and/or CO₂, and wherein separating components of the OCM product mixture excludes cryogenic distillation, (d) introducing benzene and at least a portion of the EB reactant mixture to an EB reactor, (e) allowing a portion of the benzene to react in a liquid phase with at least a portion of the ethylene of the EB reactant mixture to form EB, (f) recovering an EB product mixture and an unreacted alkanes mixture from the EB reactor, wherein the EB product mixture comprises EB and unreacted benzene, and wherein the unreacted alkanes mixture comprises C₂H₆ and unreacted methane, and optionally CO and/or CO₂, and (g) optionally recycling at least a portion of the unreacted alkanes mixture to the OCM reactor.

### BRIEF DESCRIPTION OF THE DRAWINGS

For a detailed description of the preferred embodiments of the disclosed methods, reference will now be made to the accompanying drawing in which:

FIG. 1 displays a schematic of a process that integrates oxidative coupling of methane with ethylbenzene production.

FIG. 2 displays a schematic of a multi-stage process that integrates oxidative coupling of methane with ethylbenzene production.

### DETAILED DESCRIPTION

Disclosed herein are methods for producing ethylbenzene (EB) comprising (a) introducing a first oxidative coupling of methane (OCM) reactant mixture to a first OCM reactor, wherein the first OCM reactant mixture comprises methane (CH₄) and oxygen (O₂); (b) allowing at least a portion of the first OCM reactant mixture to react via an OCM reaction to form a first OCM product mixture, wherein the first OCM product mixture comprises ethylene (C₂H₄), ethane (C₂H₆), water, carbon monoxide (CO), carbon dioxide (CO₂) and unreacted methane; (c) separating components of the first OCM product mixture, wherein separating components comprises removing at least a portion of the water and optionally at least a portion of the CO and/or CO₂ from the first OCM product mixture to yield a first EB reactant mixture, and wherein the first EB reactant mixture comprises C₂H₄, C₂H₆, unreacted methane, and optionally CO and/or CO₂; (d) introducing benzene and at least a portion of the first EB reactant mixture to a first EB reactor; (e) allowing a portion of the benzene to react with at least a portion of the ethylene of the first EB reactant mixture to form EB; (f) recovering a first EB product mixture and a first unreacted alkanes mixture from the first EB reactor, wherein the first EB product mixture comprises EB and unreacted benzene, and wherein the first unreacted alkanes mixture comprises C₂H₆ and unreacted methane, and optionally CO and/or CO₂; (g) introducing O₂ and at least a portion of the first unreacted alkanes mixture to a second OCM reactor; (h) allowing at least a portion of the O₂ and at least a portion of the first unreacted alkanes mixture to react via an OCM reaction to form a second OCM product mixture, wherein the second OCM product mixture comprises C₂H₄, C₂H₆, water, CO, CO₂ and unreacted methane, and wherein an amount of unreacted methane in the second OCM product mixture is less than an amount of unreacted methane in the first OCM product mixture; (i) separating components of the second OCM product mixture, wherein separating components comprises removing at least a portion of the water and optionally at least a portion of the CO and/or CO₂ from the second OCM product mixture to yield a second EB reactant mixture, wherein the second EB reactant mixture comprises C₂H₄, C₂H₆, unreacted methane, and optionally CO and/or CO₂, and wherein an amount of unreacted methane in the second EB reactant mixture is less than an amount of unreacted methane in the first EB reactant mixture; (j) introducing at least a portion of the first EB product mixture and at least a portion of the second EB reactant mixture to a second EB reactor; (k) allowing a portion of the benzene of the first EB product mixture to react with at least a portion of the ethylene of the second EB reactant mixture to form EB; (1) recovering a second EB product mixture and a second unreacted alkanes mixture from the second EB reactor, wherein the second EB product mixture comprises EB and unreacted benzene, wherein an amount of unreacted benzene in the second EB product mixture is less than an amount of unreacted benzene in the first EB product mixture, wherein the second unreacted alkanes mixture comprises C₂H₆ and unreacted methane, and optionally CO and/or CO₂, and wherein an amount of unreacted methane in the second unreacted alkanes mixture is less than an amount of unreacted methane in the first unreacted alkanes mixture; and (m) optionally recycling at least a portion of the second unreacted alkanes to the first OCM reactor and/or the second OCM reactor. In an embodiment, producing EB can be a multi-stage process, wherein a first stage comprises steps (a) through (f), and wherein a second stage comprises steps (g) through (m). In such embodiment, the first stage and/or the second stage can be repeated as necessary to achieve a target methane conversion for the overall multi-stage process.

Other than in the operating examples or where otherwise indicated, all numbers or expressions referring to quantities of ingredients, reaction conditions, and the like, used in the specification and claims are to be understood as modified in all instances by the term “about.” Various numerical ranges are disclosed herein. Because these ranges are continuous, they include every value between the minimum and maximum values. The endpoints of all ranges reciting the same characteristic or component are independently combinable and inclusive of the recited endpoint. Unless expressly indicated otherwise, the various numerical ranges specified in this application are approximations. The endpoints of all ranges directed to the same component or property are inclusive of the endpoint and independently combinable. The term “from more than 0 to an amount” means that the named component is present in some amount more than 0, and up to and including the higher named amount.

The terms “a,” “an,” and “the” do not denote a limitation of quantity, but rather denote the presence of at least one of the referenced item. As used herein the singular forms “a,” “an,” and “the” include plural referents.

As used herein, “combinations thereof” is inclusive of one or more of the recited elements, optionally together with a like element not recited, e.g., inclusive of a combination of one or more of the named components, optionally with one or more other components not specifically named that have essentially the same function. As used herein, the term “combination” is inclusive of blends, mixtures, alloys, reaction products, and the like.

Reference throughout the specification to “an embodiment,” “another embodiment,” “other embodiments,” “some embodiments,” and so forth, means that a particular element (e.g., feature, structure, property, and/or characteristic) described in connection with the embodiment is included in at least an embodiment described herein, and may or may not be present in other embodiments. In addition, it is to be understood that the described element(s) can be combined in any suitable manner in the various embodiments.

As used herein, the terms “inhibiting” or “reducing” or “preventing” or “avoiding” or any variation of these terms, include any measurable decrease or complete inhibition to achieve a desired result.

As used herein, the term “effective,” means adequate to accomplish a desired, expected, or intended result.

As used herein, the terms “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”) are inclusive or open-ended and do not exclude additional, unrecited elements or method steps.

Unless defined otherwise, technical and scientific terms used herein have the same meaning as is commonly understood by one of skill in the art.

Compounds are described herein using standard nomenclature. For example, any position not substituted by any indicated group is understood to have its valency filled by a bond as indicated, or a hydrogen atom. A dash (“-”) that is not between two letters or symbols is used to indicate a point of attachment for a substituent. For example, —CHO is attached through the carbon of the carbonyl group.

In an embodiment, a method for producing ethylbenzene (EB) can comprise multiple stages (e.g., as part of a multi-stage process), wherein each individual stage can comprise an oxidative coupling of methane (OCM) reactor and an EB reactor in series, wherein a portion of an OCM product mixture can be introduced to the EB reactor as an EB reactant mixture, and wherein each individual stage can be repeated as necessary to achieve a target methane conversion for the overall multi-stage process. While the current disclosure will be discussed in detail in the context of a multi-stage process comprising 2 stages, it should be understood that any suitable number of stages can be used, such as for example, 2 stages, 3 stages, 4 stages, 5 stages, 6 stages, 7 stages, 8 stages, 9 stages, 10 stages, or more stages.

In an embodiment, a method for producing EB can comprise a first stage and a second stage, wherein the first stage comprises a first OCM reactor and a first EB reactor, and wherein the second stage comprises a second OCM reactor and a second EB reactor.

In an embodiment, a method for producing EB can comprise introducing a first OCM reactant mixture to a first OCM reactor, wherein the first OCM reactant mixture comprises methane (CH₄) and oxygen (O₂); and allowing at least a portion of the first OCM reactant mixture to react via an OCM reaction to form a first OCM product mixture, wherein the first OCM product mixture comprises ethylene (C₂H₄), ethane (C₂H₆), water, carbon monoxide (CO), carbon dioxide (CO₂) and unreacted methane.

The OCM has been the target of intense scientific and commercial interest for more than thirty years due to the tremendous potential of such technology to reduce costs, energy, and environmental emissions in the production of ethylene (C₂H₄). As an overall reaction, in the OCM, CH₄ and O₂ react exothermically to form C₂H₄, water (H₂O) and heat.

Generally, in the OCM, CH₄ is first oxidatively converted into ethane (C₂H₆), and then into C₂H₄. CH₄ is activated heterogeneously on a catalyst surface, forming methyl free radicals (e.g., CH₃.), which then couple in a gas phase to form C₂H₆. C₂H₆ subsequently undergoes dehydrogenation to form C₂H₄. An overall yield of desired C₂ hydrocarbons is reduced by non-selective reactions of methyl radicals with the catalyst surface and/or oxygen in the gas phase, which produce (undesirable) carbon monoxide and carbon dioxide. Some of the best reported OCM outcomes encompass a ˜20% conversion of methane and ˜80% selectivity to desired C₂ hydrocarbons.

In some embodiments, an OCM reactor (e.g. the first OCM reactor, the second OCM reactor) can comprise an OCM catalyst. In such embodiments, the OCM catalyst can comprise basic oxides; mixtures of basic oxides; redox elements; redox elements with basic properties; mixtures of redox elements with basic properties; mixtures of redox elements with basic properties promoted with alkali and/or alkaline earth metals; rare earth metal oxides; mixtures of rare earth metal oxides; mixtures of rare earth metal oxides promoted by alkali and/or alkaline earth metals; manganese; manganese compounds; lanthanum; lanthanum compounds; sodium; sodium compounds; cesium; cesium compounds; calcium; calcium compounds; and the like; or combinations thereof.

Nonlimiting examples of OCM catalysts suitable for use in the present disclosure include CaO, MgO, BaO, CaO—MgO, CaO—BaO, Li/MgO, MnO₂, W₂O₃, SnO₂, MnO₂—W₂O₃, MnO₂—W₂O₃—Na₂O, MnO₂—W₂O₃—Li₂O, La₂O₃, SrO/La₂O₃, CeO₂, Ce₂O₃, La/MgO, La₂O₃—CeO₂, La₂O₃—CeO₂—Na₂O, La₂O₃—CeO₂—CaO, Na—Mn—La₂O₃/Al₂O₃, Na—Mn—O/SiO₂, Na₂WO₄—Mn/SiO₂, Na₂WO₄—Mn—O/SiO₂, and the like, or combinations thereof. In an embodiment, catalytic OCM processes and reactors (e.g., an OCM reactor, a first OCM reactor, a second OCM reactor, etc.) are described in more detail in U.S. Provisional Application No. 62/209,561 and U.S. Provisional Application No. 62/183,453, each of which is incorporated by reference herein in its entirety.

Oxidation of methane at high temperatures (e.g., from about 700° C. to about 1,100° C.) can lead to the appearance of the following reactions, as shown in reactions (1)-(4):

2CH₄+O₂=C₂H₄+2H₂O,ΔH=−67 kcal/mol (1)

2CH₄+1/2O₂=C₂H₆+H₂O,ΔH=−42 kcal/mol (2)

CH₄+1.5O₂=CO+2H₂O,ΔH=−124 kcal/mol (3)

CH₄+2O₂=CO₂+2H₂OΔH=−192 kcal/mol (4)

Generally, in the absence of an OCM catalyst, conversion of methane is low and the main products of conversion are CO and CO₂, as thermodynamically favored by reactions (3) and (4).

In an embodiment, the OCM reaction can be conducted in the absence of an OCM catalyst, by controlling a range of reaction temperature, a reaction residence time and a reaction feed composition (e.g., a reactant mixture composition) in such a way to maximize a C₂₊ selectivity and the production of a high H₂/CO molar ratio (e.g., from about 0.3:1 to about 2:1), thereby minimizing CO₂ formation by reaction (4). In some embodiments, controlling a reaction feed composition can further comprise introducing to the reactor (e.g., a non-catalytic OCM reactor) other components (e.g., reagents other than methane and oxygen), such as for example hydrogen, thereby changing the pathway of methane conversion reactions.

In some embodiments, an OCM reactor (e.g. the first OCM reactor, the second OCM reactor) can exclude an OCM catalyst. In such embodiments, the OCM reactor (e.g. the first OCM reactor, the second OCM reactor) can be characterized by an OCM reactor temperature of from about 700° C. to about 1,100° C., alternatively from about 750° C. to about 1,050° C., alternatively from about 800° C. to about 1,025° C., or alternatively from about 950° C. to about 1,000° C. In an embodiment, non-catalytic OCM processes and reactors (e.g., an OCM reactor, a first OCM reactor, a second OCM reactor, etc.) are described in more detail in U.S. Provisional Application No. 62/183,456, which is incorporated by reference herein in its entirety.

As will be appreciated by one of skill in the art, and with the help of this disclosure, when more than one OCM reactor is used (as is the case in a multi-stage process), all OCM reactors can be catalytic; alternatively, all OCM reactors can be non-catalytic; or alternatively, some OCM reactors can be catalytic, while some other OCM reactors can be non-catalytic.

In an embodiment, the first OCM reactant mixture can comprise a hydrocarbon or mixtures of hydrocarbons, and oxygen. In some embodiments, the hydrocarbon or mixtures of hydrocarbons can comprise natural gas (e.g., CH₄), liquefied petroleum gas comprising C₂-C₅ hydrocarbons, C₆₊ heavy hydrocarbons (e.g., C₆ to C₂₄ hydrocarbons such as diesel fuel, jet fuel, gasoline, tars, kerosene, etc.), oxygenated hydrocarbons, biodiesel, alcohols, dimethyl ether, and the like, or combinations thereof. In an embodiment, the reactant mixture can comprise CH₄ and O₂. As will be appreciated by one of skill in the art, and with the help of this disclosure, methane (or a hydrocarbon or mixtures of hydrocarbons) is introduced into a multi-stage process in the first stage into the OCM reactor (e.g., a first OCM reactor); the OCM reactant mixture for subsequent stages (e.g., a second stage) will utilize the unreacted methane and any other hydrocarbons present that were recovered from the first stage (after passing through any other processes that are part of the first stage). In some embodiments, some methane (or a hydrocarbon or mixtures of hydrocarbons) could be optionally added to reactant mixtures in stages other than the first stage (e.g., fresh hydrocarbon feed at one or more stages subsequent to a first stage), to supplement a recovered unreacted methane, if necessary.

In an embodiment, the O₂ used in the first OCM reactant mixture and/or in any subsequent stages in any OCM reactor (e.g., a second OCM reactor), can be oxygen gas (which may be obtained via a membrane separation process), technical oxygen (which may contain some air), air, oxygen enriched air, and the like, or combinations thereof.

In an embodiment, the first OCM reactant mixture can further comprise a diluent. A diluent can also be introduced in any subsequent stages in any OCM reactor (e.g., a second OCM reactor). The diluent is inert with respect to the OCM reaction, e.g., the diluent does not participate in the OCM reaction. In an embodiment, the diluent can comprise water, nitrogen, inert gases, and the like, or combinations thereof. In an embodiment, the diluent can be present in the OCM reactant mixture (e.g., first OCM reactant mixture, second OCM reactant mixture) in an amount of from about 0.5% to about 80%, alternatively from about 5% to about 50%, or alternatively from about 10% to about 30%, based on the total volume of the OCM reactant mixture. In an embodiment, the use of a diluent in an OCM process is described in more detail in U.S. Provisional Application No. 62/209,561.

In an embodiment, a method for producing EB can comprise separating components of the first OCM product mixture, wherein separating components comprises removing at least a portion of the water and optionally at least a portion of the CO and/or CO₂ from the first OCM product mixture to yield a first EB reactant mixture, wherein the first OCM product mixture comprises C₂H₄, C₂H₆, water, CO, CO₂, and unreacted methane, and wherein the first EB reactant mixture comprises C₂H₄, C₂H₆, unreacted methane, and optionally CO and/or CO₂.

In an embodiment, separating components of an OCM product mixture (e.g., the first OCM product mixture, the second OCM product mixture) excludes cryogenic distillation or separation.

In some embodiments, a method for producing EB can comprise separating or removing water from the first OCM product mixture, to yield the first EB reactant mixture comprising C₂H₄, C₂H₆, unreacted methane, CO and CO₂.

In an embodiment, at least a portion of the water can be removed from the first OCM product mixture, to yield a first EB reactant mixture. In an embodiment, the first OCM product mixture can be introduced to a compressor, and then to a water quench vessel. Generally, compressing a gas that contains water from a first pressure to a second pressure (wherein the second pressure is greater than the first pressure) will lead to the water condensing at the second pressure at an increased temperature as compared to a temperature where water of an otherwise similar gas condenses at the first pressure. In an embodiment, the compressed first OCM product mixture can be further cooled in a cooling tower or in the water quench vessel to promote water condensation and removal.

In an embodiment, the first EB reactant mixture comprising C₂H₄, C₂H₆, unreacted methane, CO and CO₂ can be further subjected to CO and/or CO₂ removal, to yield a first EB reactant mixture comprising C₂H₄, C₂H₆, and unreacted methane.

In an embodiment, at least a portion of CO₂ can be removed from the first EB reactant mixture by using a CO₂ separator. In some embodiments, the CO₂ separator can comprise CO₂ removal by amine (e.g., monoethanolamine) absorption (e.g., amine scrubbing), pressure swing adsorption, temperature swing adsorption, gas separation membranes (e.g., porous inorganic membranes, palladium membranes, polymeric membranes, zeolites, etc.), and the like, or combinations thereof. In an embodiment, the CO₂ separator can comprise CO₂ removal by amine absorption.

In an embodiment, at least a portion of CO can be removed from the first EB reactant mixture. In some embodiments, the CO can be converted into CO₂ (possibly prior to removal of at least a portion of CO₂ from the first EB reactant mixture), for example by catalytic oxidation the presence of a metal (e.g., Pt, Pd, etc.) based catalyst.

In an embodiment, an EB reactant mixture (e.g., first EB reactant mixture, second EB reactant mixture) can be characterized by an EB reactant mixture temperature of from about 100° C. to about 270° C., alternatively from about 125° C. to about 225° C., or alternatively from about 150° C. to about 250° C. In an embodiment, the method for producing EB can exclude cooling the EB reactant mixture (e.g., first EB reactant mixture, second EB reactant mixture) prior to introducing the EB reactant mixture to the EB reactor. As will be appreciated by one of skill in the art, and with the help of this disclosure, the reaction for producing EB can run at temperatures of from about 100° C. to about 270° C., and as such an EB reactant mixture (e.g., first EB reactant mixture, second EB reactant mixture) that is introduced to an EB reactor does not require cooling, and can be used at the temperature it has upon separating components from the OCM product mixture (e.g., first OCM product mixture, second OCM product mixture).

In an embodiment, a method for producing EB can comprise introducing benzene and at least a portion of the first EB reactant mixture to a first EB reactor; and allowing a portion of the benzene to react with at least a portion of the ethylene of the first EB reactant mixture to form EB.

In some embodiments, an EB reactor (e.g., first EB reactor, the second EB reactor) can be characterized by an EB reactor temperature of from about 100° C. to about 270° C., alternatively from about 125° C. to about 225° C., or alternatively from about 150° C. to about 250° C.

In an embodiment, the benzene can react in a liquid phase in an EB liquid phase reactor with at least a portion of the ethylene of the EB reactant mixture (e.g., first EB reactant mixture, second EB reactant mixture) to form EB. In some embodiments the EB liquid phase reactor comprises a catalytic distillation (CD) reactor or a CD column. The CD column combines reaction and fractionation in a single unit operation. Alkylation reaction (e.g., EB formation reaction) can take place isothermally, and at low temperature (below about 290° C., which is the critical temperature of benzene). Reaction products can be continually removed from a reaction zone by distillation. As such, the formation of by-product impurities can be limited, and product purity and yields can be enhanced. Low operating temperatures (below about 290° C.) can result in lower operating pressures, which can minimize fugitive emissions. All heat input, including the heat of alkylation reaction, can be recovered as useful steam.

In embodiments where liquid phase reactors or CD columns are used for EB production by catalytic alkylation of benzene with ethylene, product yields of up to 99.9% can be obtained, and an EB purity of up to 99.9% can be achieved. In such embodiments, xylene impurity formation can be virtually eliminated, avoiding further EB processing difficulties.

In an embodiment, an EB liquid phase reactor (e.g., the first EB reactor, the second EB reactor) can be characterized by an EB reactor pressure of from about 150 psig to about 750 psig, alternatively from about 200 psig to about 700 psig, or alternatively from about 250 psig to about 650 psig.

In some embodiments, the EB reactant mixture (e.g., first EB reactant mixture, second EB reactant mixture) can be pressurized prior to introducing to the EB reactor (e.g., the first EB reactor, the second EB reactor). As will be appreciated by one of skill in the art, and with the help of this disclosure, if the pressure inside the reactor is greater than the pressure of the EB reactant mixture, the EB reactant mixture could be pressurized to achieve an EB reactant mixture pressure that is about the same as the pressure inside the reactor.

In an embodiment, an EB liquid phase reactor (e.g., the first EB reactor, the second EB reactor) can comprise a catalyst that is active for the alkylation of benzene with ethylene, such as for example a zeolite (e.g., an acidic zeolite catalyst) or a Lewis acid catalyst (e.g., boron compounds, aluminum halides, etc.). Nonlimiting examples of zeolite catalysts suitable for use in the present disclosure for the alkylation reaction for producing EB include acidic zeolite/alumina, Y-zeolite/alumina, dealuminized mordenite, alumina/magnesium silicate, zeolite beta/alumina, any other suitable acidic zeolite catalysts, any other suitable molecular sieve catalysts, and the like, or combinations thereof.

In an embodiment, the benzene can react in a gas phase in an EB gas phase reactor with at least a portion of the ethylene of the EB reactant mixture (e.g., first EB reactant mixture, second EB reactant mixture) to form EB. In some embodiments, the EB gas phase reactor can comprise a zeolite catalyst.

In some embodiments, the EB gas phase reactor (e.g., the first EB reactor, the second EB reactor) can comprise one or more fixed catalytic beds, such as for example fixed zeolite beds. The purity of the products obtained in EB gas phase reactors is lower than a product purity obtained in CD columns in liquid phase. In gas phase, about 15% of the produced EB reacts further with ethylene to form di-ethylbenzene isomers, tri-ethylbenzene isomers, and other heavier aromatic products, as well as xylenes. Generally, xylenes are considered undesirable in EB, when EB is further used for styrene production, xylenes are considered an impurity in the styrene.

In some embodiments, the benzene can react in a mixed liquid-gas phase in an EB mixed phase reactor with at least a portion of the ethylene of the EB reactant mixture (e.g., first EB reactant mixture, second EB reactant mixture) to form EB.

As will be appreciated by one of skill in the art, and with the help of this disclosure, when more than one EB reactor is used (as is the case in a multi-stage process), all EB reactors can be liquid phase reactors; alternatively, all EB reactors can be gas phase reactors; or alternatively, some EB reactors can be liquid phase reactors, while some other EB reactors can be gas phase reactors.

In an embodiment, a method for producing EB can comprise recovering a first EB product mixture and a first unreacted alkanes mixture from the first EB reactor, wherein the first EB product mixture comprises EB and unreacted benzene, and wherein the first unreacted alkanes mixture comprises C₂H₆ and unreacted methane, and optionally CO and/or CO₂.

In an embodiment, at least a portion of the EB can be recovered from the EB product mixture (e.g., the first EB product mixture, the second EB product mixture) by any suitable methodology, such as for example by distillation.

In an embodiment, at least a portion of the benzene can be recovered from the EB product mixture (e.g., the first EB product mixture, the second EB product mixture) to yield recovered benzene, by any suitable methodology, such as for example by distillation. In such embodiment, at least a portion of the recovered benzene can be recycled to the EB reactor (e.g., the first EB reactor, the second EB reactor).

In an embodiment, an unreacted alkanes mixture (e.g., the first unreacted alkanes mixture, the second unreacted alkanes mixture) can comprise less than about 0.05%, alternatively less than about 0.04%, alternatively less than about 0.03%, alternatively less than about 0.02%, or alternatively less than about 0.01% ethylene. As will be appreciated by one of skill in the art, and with the help of this disclosure, in some instances, virtually all ethylene present in the feed to the EB reactor will react during the benzene alkylation reaction.

In an embodiment, a method for producing EB can comprise a second stage, wherein the second stage comprises a second OCM reactor and a second EB reactor. In such embodiment, a method for producing EB can comprise introducing O₂ and at least a portion of the first unreacted alkanes mixture to a second OCM reactor; allowing at least a portion of the O₂ and at least a portion of the first unreacted alkanes mixture to react via an OCM reaction to form a second OCM product mixture, wherein the second OCM product mixture can comprise C₂H₄, C₂H₆, water, CO, CO₂ and unreacted methane, and wherein an amount of unreacted methane in the second OCM product mixture can be less than an amount of unreacted methane in the first OCM product mixture, with the proviso that no fresh or supplemental methane is added to the second stage to desirably produce an increase in a methane concentration; separating components of the second OCM product mixture, wherein separating components can comprise removing at least a portion of the water and optionally at least a portion of the CO and/or CO₂ from the second OCM product mixture to yield a second EB reactant mixture, wherein the second EB reactant mixture can comprise C₂H₄, C₂H₆, unreacted methane, and optionally CO and/or CO₂, and wherein an amount of unreacted methane in the second EB reactant mixture can be less than an amount of unreacted methane in the first EB reactant mixture; introducing at least a portion of the first EB product mixture and at least a portion of the second EB reactant mixture to a second EB reactor; allowing a portion of the benzene of the first EB product mixture to react with at least a portion of the ethylene of the second EB reactant mixture to form EB; recovering a second EB product mixture and a second unreacted alkanes mixture from the second EB reactor, wherein the second EB product mixture can comprise EB and unreacted benzene, wherein an amount of unreacted benzene in the second EB product mixture can be less than an amount of unreacted benzene in the first EB product mixture, with the proviso that no fresh or supplemental benzene is added to the second stage to desirably produce an increase in a benzene concentration, wherein the second unreacted alkanes mixture can comprise C₂H₆ and unreacted methane, and optionally CO and/or CO₂, and wherein an amount of unreacted methane in the second unreacted alkanes mixture can be less than an amount of unreacted methane in the first unreacted alkanes mixture, with the proviso that no fresh or supplemental methane is added to the second stage to desirably produce an increase in a methane concentration; and optionally recycling at least a portion of the second unreacted alkanes to the first OCM reactor and/or the second OCM reactor. For purposes of the disclosure herein, all descriptions related to the first stage (such as descriptions of reactors, OCM reactor, EB reactor, reactant mixtures, EB reactant mixture, OCM reactant mixture, product mixture, OCM product mixture, EB product mixture, unreacted alkanes mixture, etc.) can be applied to the corresponding components of the second stage (such as descriptions of reactors, OCM reactor, EB reactor, reactant mixtures, EB reactant mixture, OCM reactant mixture, product mixture, OCM product mixture, EB product mixture, unreacted alkanes mixture, etc., respectively), unless otherwise specified herein.

As will be appreciated by one of skill in the art, and with the help of this disclosure, in some instances, the methane reacting in the second stage in the second OCM reactor is primarily methane that was introduced to the first OCM reactor, didn't react, and was subsequently recovered as unreacted methane (as part of the first unreacted alkanes mixture), with the proviso that no fresh or supplemental methane was added to the second stage to desirably produce an increase in a methane concentration. Further, as will be appreciated by one of skill in the art, and with the help of this disclosure, when fresh methane is introduced to the second stage, an amount of unreacted methane recovered from the second stage (as part of the second unreacted alkanes mixture) minus the amount of fresh methane introduced to the second stage is less than the amount of unreacted methane that was recovered from the first stage (as part of the first unreacted alkanes mixture) and was subsequently introduced to the second stage. In some embodiments, a method for producing EB can further comprise introducing additional CH₄ to the second OCM reactor.

As will be appreciated by one of skill in the art, and with the help of this disclosure, in some instances, the benzene reacting in the second stage in the second EB reactor is primarily benzene that was introduced to the first EB reactor, didn't react, and was subsequently recovered as unreacted benzene (as part of the first EB product mixture), with the proviso that no fresh or supplemental benzene was added to the second stage to desirably produce an increase in a benzene concentration. Further, as will be appreciated by one of skill in the art, and with the help of this disclosure, when fresh benzene is introduced to the second stage, an amount of unreacted benzene recovered from the second stage (as part of the second EB product mixture) minus the amount of fresh benzene introduced to the second stage is less than the amount of unreacted benzene that was recovered from the first stage (as part of the first EB product mixture) and was subsequently introduced to the second stage. In some embodiment, a method for producing EB can further comprise introducing additional benzene to the second EB reactor.

In some embodiments, at least a portion of the second unreacted alkanes mixture (or an unreacted alkanes mixture recovered from a last stage of the multi-stage process) can be used as a source of fuel for generating energy.

In an embodiment, an yield to EB in a multi-stage process can be from about 90% to about 100%, alternatively from about 90.5% to about 99.9%, or alternatively from about 91% to about 99.8%. Generally, an yield to a certain product can be calculated by dividing the actual yield by the theoretical yield. For purposes of the disclosure herein, a theoretical yield to EB is based on the amount of ethylene introduced to the reactor, as the benzene is introduced in excess.

In an embodiment, a methane conversion in a multi-stage process can be from about 5% to about 100%, alternatively from about 25% to about 95%, or alternatively from about 50% to about 90%. Generally, a conversion of a reagent or reactant refers to the percentage (usually mol %) of reagent that reacted to both undesired and desired products, based on the total amount (e.g., moles) of reagent present before any reaction took place. For example, the methane conversion in a multi-stage process can be calculated by using equation (5):

$$Methane multi \text{-} stage conversion = \frac{{Moles}_{{CH}_{4}}^{in multi \text{-} stage process} - {Moles}_{{CH}_{4}}^{out multi \text{-} stage process}}{{Moles}_{{CH}_{4}}^{in mult i \text{-} stage process}} \times 100 \% \qquad ( 5 )$$

wherein Moles_{CH}_{₄}^{inmulti-stage process}=number of moles of methane that was introduced to the multi-stage process; and Moles_{CH}_{₄}^{in multi-stage process}=number of moles of CH₄ that was recovered from the multi-stage process.

In an embodiment, equal to or greater than about 5 mol %, alternatively equal to or greater than about 10 mol %, or alternatively equal to or greater than about 15 mol % of the methane in the first OCM reactant mixture can be converted overall to EB.

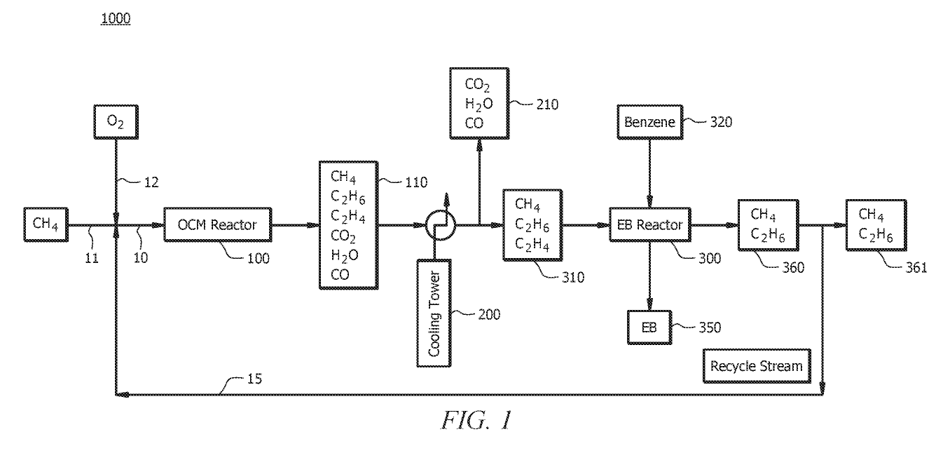

In some embodiments, the method for producing ethylbenzene (EB) can comprise a single stage process (for example as shown in FIG. 1), wherein the single stage can comprise an oxidative coupling of methane (OCM) reactor and an EB reactor in series, and wherein a portion of an OCM product mixture can be introduced to the EB reactor as an EB reactant mixture.

For purposes of the disclosure herein, all descriptions related to any stage of the multi-stage process (such as descriptions of reactors, OCM reactor, EB reactor, reactant mixtures, EB reactant mixture, OCM reactant mixture, product mixture, OCM product mixture, EB product mixture, unreacted alkanes mixture, etc.) can be applied to the corresponding components of the single stage process (such as descriptions of reactors, OCM reactor, EB reactor, reactant mixtures, EB reactant mixture, OCM reactant mixture, product mixture, OCM product mixture, EB product mixture, unreacted alkanes mixture, etc., respectively), and vice-versa, unless otherwise specified herein.

Referring to the embodiment of FIG. 1, an EB production system 1000 is disclosed. The EB production system 1000 generally comprises an OCM reactor 100; a cooling tower 200; and an EB reactor 300. As will be appreciated by one of skill in the art, and with the help of this disclosure, EB production system components can be in fluid communication with each other through any suitable conduits (e.g., pipes, streams, etc.).

In an embodiment, a method for producing EB can comprise one or more stages, for example a stage comprising (a) introducing an OCM reactant mixture stream 10 to the OCM reactor 100, wherein the OCM reactant mixture can comprise CH₄ (supplied by CH₄ stream 11) and O₂ (supplied by O₂ stream 12); (b) allowing at least a portion of the OCM reactant mixture to react via an OCM reaction to form an OCM product mixture 110, wherein the OCM product mixture 110 can comprise C₂H₄, C₂H₆, water, CO, CO₂, and unreacted methane; (c) separating at least a portion of the water and optionally at least a portion of the CO and/or CO₂ 210 from the OCM product mixture 110 to yield an EB reactant mixture 310, such as for example by introducing the OCM product mixture 110 to the cooling tower 200, wherein the EB reactant mixture 310 can comprise C₂H₄, C₂H₆, unreacted methane, and optionally CO and/or CO₂; (d) introducing benzene 320 and at least a portion of the EB reactant mixture 310 to the EB reactor 300, wherein the at least a portion of the EB reactant mixture 310 can be pressurized prior to introducing to the EB reactor 300; (e) allowing a portion of the benzene 320 to react in a liquid phase with at least a portion of the ethylene of the EB reactant mixture 310 to form EB; (f) recovering an EB product mixture 350 and an unreacted alkanes mixture 360 from the EB reactor 300, wherein the EB product mixture 350 can comprise EB and unreacted benzene, and wherein the unreacted alkanes mixture 360 can comprise C₂H₆ and unreacted methane, and optionally CO and/or CO₂; and (g) optionally recycling at least a portion of the unreacted alkanes mixture 360 as recycled alkanes stream 15 to the OCM reactor 100. In an embodiment, the EB reactor in a single stage process for producing EB, such as shown in FIG. 1, is a liquid phase reactor. In an alternative embodiment, all or a portion of unreacted alkanes mixture 360, 361 can be fed to a second stage, wherein the second stage is substantially similar to the first stage.

In an embodiment, an yield to EB in a single-stage process can be from about 90% to about 100%, alternatively from about 90.5% to about 99.9%, or alternatively from about 91% to about 99.8%.

In an embodiment, a methane conversion in a single-stage process can be from about 5% to about 100%, alternatively from about 25% to about 95%, or alternatively from about 50% to about 90%. For example, the methane conversion in a single-stage process can be calculated by using equation (6):

$$Methane sin gle \text{-} stage conversion = \frac{{Moles}_{{CH}_{4}}^{in } - {Moles}_{{CH}_{4}}^{out }}{{Moles}_{{CH}_{4}}^{in}} \times 100 \% \qquad ( 6 )$$

wherein Moles_{CH}_{₄}ⁱⁿ=number of moles of methane that was introduced to the single-stage process (e.g., number of moles of methane that was introduced to the OCM reactor); and Moles_{CH}_{₄}^{out}=number of moles of CH₄ that was recovered from the single-stage process (e.g., number of moles of methane that was recovered from the OCM reactor).

In an embodiment, equal to or greater than about 5 mol %, alternatively equal to or greater than about 10 mol %, or alternatively equal to or greater than about 15 mol % of the methane in the OCM reactant mixture can be converted overall to EB.

As will be appreciated by one of sill in the art, and with the help of this disclosure, while the current disclosure is discussed in detail in the context of producing EB from ethylene from an OCM process, the ethylene from the OCM process could be also used for the production of other ethylene derivatives (EDs), such as for example ethylene oxide, vinyl chloride, etc.

In an embodiment, a method for producing an ED can comprise (a) introducing an OCM reactant mixture to an OCM reactor, wherein the OCM reactant mixture can comprise CH₄ and O₂; (b) allowing at least a portion of the OCM reactant mixture to react via an OCM reaction to form an OCM product mixture, wherein the OCM product mixture can comprise C₂H₄, C₂H₆, water, CO, CO₂, and unreacted methane; (c) separating at least a portion of the water and optionally at least a portion of the CO and/or CO₂ from the OCM product mixture to yield an ED reactant mixture, wherein the ED reactant mixture can comprise C₂H₄, C₂H₆, unreacted methane, and optionally CO and/or CO₂; (d) introducing at least a portion of the ED reactant mixture to an ED reactor; (e) allowing at least a portion of the ethylene of the ED reactant mixture to react and form the ED; and (f) recovering an ED product mixture from the ED reactor, wherein the ED product mixture can comprise the ED. For example, in FIG. 1, the EB reactor 300 could be replaced with an ED reactor, and ED products could be recovered.

In an embodiment, the ED can be selected from the group consisting of ethylbenzene, ethylene oxide, and vinyl chloride.

In an embodiment, a method for producing EB can comprise (a) introducing an OCM reactant mixture to an OCM reactor, wherein the OCM reactant mixture can comprise CH₄ and O₂; (b) allowing at least a portion of the OCM reactant mixture to react via an OCM reaction to form an OCM product mixture, wherein the OCM product mixture can comprise C₂H₄, C₂H₆, water, CO, CO₂, and unreacted methane; (c) separating components of the OCM product mixture, wherein separating components can comprise removing at least a portion of the water and optionally at least a portion of the CO and/or CO₂ from the OCM product mixture to yield an EB reactant mixture, wherein the EB reactant mixture can comprise C₂H₄, C₂H₆, unreacted methane, and optionally CO and/or CO₂, and wherein separating components of the OCM product mixture excludes cryogenic distillation; (d) introducing benzene and at least a portion of the EB reactant mixture to an EB reactor; (e) allowing a portion of the benzene to react in a liquid phase with at least a portion of the ethylene of the EB reactant mixture to form EB; (f) recovering an EB product mixture and an unreacted alkanes mixture from the EB reactor, wherein the EB product mixture can comprise EB and unreacted benzene, and wherein the unreacted alkanes mixture can comprise C₂H₆ and unreacted methane, and optionally CO and/or CO₂; and (g) optionally recycling at least a portion of the unreacted alkanes mixture to the OCM reactor.

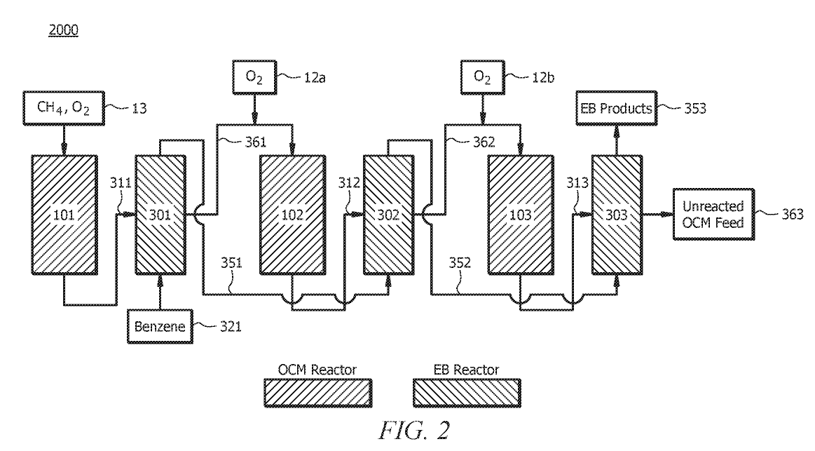

Referring to the embodiment of FIG. 2, an EB production system 2000 is disclosed. The EB production system 2000 generally comprises a first OCM reactor 101; a second OCM reactor 102; a third OCM reactor 103; a first EB reactor 301; a second EB reactor 302; and a third EB reactor 303. As will be appreciated by one of skill in the art, and with the help of this disclosure, EB production system components can be in fluid communication with each other through any suitable conduits (e.g., pipes, streams, etc.). For purposes of the disclosure herein, all descriptions related to any stage of a multi-stage process (such as descriptions of reactors, OCM reactor, EB reactor, reactant mixtures, EB reactant mixture, OCM reactant mixture, product mixture, OCM product mixture, EB product mixture, unreacted alkanes mixture, etc.) previously disclosed herein can be applied to the corresponding components of FIG. 2 (such as descriptions of reactors, OCM reactor, EB reactor, reactant mixtures, EB reactant mixture, OCM reactant mixture, product mixture, OCM product mixture, EB product mixture, unreacted alkanes mixture, etc., respectively), and vice-versa, unless otherwise specified herein. Further, as will be appreciated by one of skill in the art, and with the help of this disclosure, while the EB production system 2000 disclosed in FIG. 2 comprises a three-stage process, it should be understood that an EB production system, such as the EB production system 2000, can comprise any suitable number of stages, such as for example, 2 stages, 3 stages, 4 stages, 5 stages, 6 stages, 7 stages, 8 stages, 9 stages, 10 stages, or more stages.

In an embodiment, a method for producing EB can comprise three stages (e.g., three-stage process as represented in the embodiment of FIG. 2), for example (i) a first stage comprising (a1) introducing a first OCM reactant mixture 13 to the first OCM reactor 101, wherein the first OCM reactant mixture 13 can comprise CH₄ and O₂, (b1) allowing at least a portion of the first OCM reactant mixture 13 to react via an OCM reaction to form a first OCM product mixture, wherein the first OCM product mixture can comprise C₂H₄, C₂H₆, water, CO, CO₂, and unreacted methane, (c1) separating at least a portion of the water and optionally at least a portion of the CO and/or CO₂ from the first OCM product mixture to yield a first EB reactant mixture, such as for example by introducing the first OCM product mixture to a cooling tower, wherein the first EB reactant mixture can comprise C₂H₄, C₂H₆, unreacted methane, and optionally CO and/or CO₂, (d1) introducing at least a portion of the first EB reactant mixture via stream 311 and benzene 321 to the first EB reactor 301, wherein the at least a portion of the first EB reactant mixture can be pressurized prior to introducing to the first EB reactor 301, (e1) allowing a portion of the benzene 321 to react with at least a portion of the ethylene of the first EB reactant mixture to form EB, and (f1) recovering a first EB product mixture via stream 351 and a first unreacted alkanes mixture via stream 361 from the first EB reactor 301, wherein the first EB product mixture can comprise EB and unreacted benzene, and wherein the first unreacted alkanes mixture can comprise C₂H₆ and unreacted methane, and optionally CO and/or CO₂; (ii) a second stage comprising (a2) introducing O₂ 12 a and at least a portion of the first unreacted alkanes mixture comprising CH₄ to the second OCM reactor 102, (b2) allowing at least a portion of the CH₄ of the first unreacted alkanes mixture and at least a portion of the O₂ 12 a to react via an OCM reaction to form a second OCM product mixture, wherein the second OCM product mixture can comprise C₂H₄, C₂H₆, water, CO, CO₂, and unreacted methane, (c2) separating at least a portion of the water and optionally at least a portion of the CO and/or CO₂ from the second OCM product mixture to yield a second EB reactant mixture, such as for example by introducing the second OCM product mixture to a cooling tower, wherein the second EB reactant mixture can comprise C₂H₄, C₂H₆, unreacted methane, and optionally CO and/or CO₂, (d2) introducing at least a portion of the second EB reactant mixture via stream 312 and at least a portion of the first EB product mixture comprising benzene to the second EB reactor 302, wherein the at least a portion of the second EB reactant mixture can be pressurized prior to introducing to the second EB reactor 302, (e2) allowing a portion of the benzene of the first EB product mixture to react with at least a portion of the ethylene of the second EB reactant mixture to form EB, and (f2) recovering a second EB product mixture via stream 352 and a second unreacted alkanes mixture via stream 362 from the second EB reactor 302, wherein the second EB product mixture can comprise EB and unreacted benzene, and wherein the second unreacted alkanes mixture can comprise C₂H₆ and unreacted methane, and optionally CO and/or CO₂; and (iii) a third stage comprising (a3) introducing O₂ 12 b and at least a portion of the second unreacted alkanes mixture comprising CH₄ to the third OCM reactor 103, (b3) allowing at least a portion of the CH₄ of the second unreacted alkanes mixture and at least a portion of the O₂ 12 b to react via an OCM reaction to form a third OCM product mixture, wherein the third OCM product mixture can comprise C₂H₄, C₂H₆, water, CO, CO₂, and unreacted methane, (c3) separating at least a portion of the water and optionally at least a portion of the CO and/or CO₂ from the third OCM product mixture to yield a third EB reactant mixture, such as for example by introducing the third OCM product mixture to a cooling tower, wherein the third EB reactant mixture can comprise C₂H₄, C₂H₆, unreacted methane, and optionally CO and/or CO₂, (d3) introducing at least a portion of the third EB reactant mixture via stream 313 and at least a portion of the second EB product mixture comprising benzene to the third EB reactor 303, wherein the at least a portion of the third EB reactant mixture can be pressurized prior to introducing to the third EB reactor 303, (e3) allowing a portion of the benzene of the second EB product mixture to react with at least a portion of the ethylene of the third EB reactant mixture to form EB, and (f3) recovering a third EB product mixture 353 and a third unreacted alkanes mixture 363 from the third EB reactor 303, wherein the third EB product mixture can comprise EB and unreacted benzene, and wherein the third unreacted alkanes mixture 363 can comprise C₂H₆ and unreacted methane, and optionally CO and/or CO₂. In such embodiment, the method for producing EB can comprise optionally recycling at least a portion of the third unreacted alkanes mixture 363 to the first OCM reactor 101. An amount of unreacted methane in the third unreacted alkanes mixture 363 can be lower than an amount of unreacted methane in the second unreacted alkanes mixture; and/or an amount of unreacted methane in the second unreacted alkanes mixture can be lower than an amount of unreacted methane in the first unreacted alkanes mixture; with the proviso that no fresh or supplemental methane is added to the second stage and/or the third stage to desirably produce an increase in a methane concentration. An amount of unreacted benzene in the third EB product mixture 353 can be lower than an amount of unreacted benzene in the second EB product mixture; and/or an amount of unreacted benzene in the second EB product mixture can be lower than an amount of unreacted benzene in the first EB product mixture; with the proviso that no fresh or supplemental benzene is added to the second stage and/or the third stage to desirably produce an increase in a benzene concentration.

In an embodiment, a method for producing EB as disclosed herein can advantageously display improvements in one or more method characteristics when compared to an otherwise similar method that does not integrate OCM with other processes for producing desired products. Generally, in an OCM process, ethylene is separated by cryogenic distillation, an energy intensive separation. The method for producing EB as disclosed herein can advantageously eliminate a cryogenic distillation step for ethylene separation, which in turn can reduce the cost of ethylene, thereby providing for the production of EB at a lower cost.

In an embodiment, a method for producing EB as disclosed herein can advantageously display an increased selectivity of OCM, especially with a multi-stage process, when compared to the selectivity of a similar OCM process that is not integrated with EB production. By selectively removing ethylene product from an OCM product stream via EB formation, ethylene doesn't get further oxidized to CO and CO2 in the following stages, and consequently a selectivity of the process is increased.

In an embodiment, a method for producing EB via a multi-stage process as disclosed herein can advantageously provide for controlling a methane conversion in each stage, so as to achieve the best selectivity for OCM, such that a total process yield can be increased. Additional advantages of the methods for the production of EB as disclosed herein can be apparent to one of skill in the art viewing this disclosure.

For the purpose of any U.S. national stage filing from this application, all publications and patents mentioned in this disclosure are incorporated herein by reference in their entireties, for the purpose of describing and disclosing the constructs and methodologies described in those publications, which might be used in connection with the methods of this disclosure. Any publications and patents discussed herein are provided solely for their disclosure prior to the filing date of the present application. Nothing herein is to be construed as an admission that the inventors are not entitled to antedate such disclosure by virtue of prior invention.

In any application before the United States Patent and Trademark Office, the Abstract of this application is provided for the purpose of satisfying the requirements of 37 C.F.R. §1.72 and the purpose stated in 37 C.F.R. §1.72(b) “to enable the United States Patent and Trademark Office and the public generally to determine quickly from a cursory inspection the nature and gist of the technical disclosure.” Therefore, the Abstract of this application is not intended to be used to construe the scope of the claims or to limit the scope of the subject matter that is disclosed herein. Moreover, any headings that can be employed herein are also not intended to be used to construe the scope of the claims or to limit the scope of the subject matter that is disclosed herein. Any use of the past tense to describe an example otherwise indicated as constructive or prophetic is not intended to reflect that the constructive or prophetic example has actually been carried out.

The present disclosure is further illustrated by the following examples, which are not to be construed in any way as imposing limitations upon the scope thereof. On the contrary, it is to be clearly understood that resort can be had to various other aspects, embodiments, modifications, and equivalents thereof which, after reading the description herein, can be suggest to one of ordinary skill in the art without departing from the spirit of the present invention or the scope of the appended claims.

### ADDITIONAL DISCLOSURE

A first embodiment, which is a method for producing ethylbenzene (EB) comprising (a) introducing a first oxidative coupling of methane (OCM) reactant mixture to a first OCM reactor, wherein the first OCM reactant mixture comprises methane (CH₄) and oxygen (O₂); (b) allowing at least a portion of the first OCM reactant mixture to react via an OCM reaction to form a first OCM product mixture, wherein the first OCM product mixture comprises ethylene (C₂H₄), ethane (C₂H₆), water, carbon monoxide (CO), carbon dioxide (CO₂) and unreacted methane; (c) separating components of the first OCM product mixture, wherein separating components comprises removing at least a portion of the water and optionally at least a portion of the CO and/or CO₂ from the first OCM product mixture to yield a first EB reactant mixture, and wherein the first EB reactant mixture comprises C₂H₄, C₂H₆, unreacted methane, and optionally CO and/or CO₂; (d) introducing benzene and at least a portion of the first EB reactant mixture to a first EB reactor; (e) allowing a portion of the benzene to react with at least a portion of the ethylene of the first EB reactant mixture to form EB; (f) recovering a first EB product mixture and a first unreacted alkanes mixture from the first EB reactor, wherein the first EB product mixture comprises EB and unreacted benzene, and wherein the first unreacted alkanes mixture comprises C₂H₆ and unreacted methane, and optionally CO and/or CO₂; (g) introducing O₂ and at least a portion of the first unreacted alkanes mixture to a second OCM reactor; (h) allowing at least a portion of the O₂ and at least a portion of the first unreacted alkanes mixture to react via an OCM reaction to form a second OCM product mixture, wherein the second OCM product mixture comprises C₂H₄, C₂H₆, water, CO, CO₂ and unreacted methane, and wherein an amount of unreacted methane in the second OCM product mixture is less than an amount of unreacted methane in the first OCM product mixture; (i) separating components of the second OCM product mixture, wherein separating components comprises removing at least a portion of the water and optionally at least a portion of the CO and/or CO₂ from the second OCM product mixture to yield a second EB reactant mixture, wherein the second EB reactant mixture comprises C₂H₄, C₂H₆, unreacted methane, and optionally CO and/or CO₂, and wherein an amount of unreacted methane in the second EB reactant mixture is less than an amount of unreacted methane in the first EB reactant mixture; (j) introducing at least a portion of the first EB product mixture and at least a portion of the second EB reactant mixture to a second EB reactor; (k) allowing a portion of the benzene of the first EB product mixture to react with at least a portion of the ethylene of the second EB reactant mixture to form EB; (1) recovering a second EB product mixture and a second unreacted alkanes mixture from the second EB reactor, wherein the second EB product mixture comprises EB and unreacted benzene, wherein an amount of unreacted benzene in the second EB product mixture is less than an amount of unreacted benzene in the first EB product mixture, wherein the second unreacted alkanes mixture comprises C₂H₆ and unreacted methane, and optionally CO and/or CO₂, and wherein an amount of unreacted methane in the second unreacted alkanes mixture is less than an amount of unreacted methane in the first unreacted alkanes mixture; and (m) optionally recycling at least a portion of the second unreacted alkanes to the first OCM reactor and/or the second OCM reactor.

A second embodiment, which is the method of the first embodiment, wherein separating components of the first OCM product mixture and/or the second OCM product mixture excludes cryogenic distillation.

A third embodiment, which is the method of any one of the first and the second embodiments excluding cooling the first EB reactant mixture and/or the second EB reactant mixture.

A fourth embodiment, which is the method of any one of the first through the third embodiments, wherein the first EB reactant mixture and/or the second EB reactant mixture are characterized by an EB reactant mixture temperature of from about 100° C. to about 270° C.

A fifth embodiment, which is the method of any one of the first through the fourth embodiments, wherein the first EB reactor and/or the second EB reactor are characterized by an EB reactor temperature of from about 100° C. to about 270° C.

A sixth embodiment, which is the method of any one of the first through the fifth embodiments, wherein the first OCM reactor and/or the second OCM reactor comprise an OCM catalyst.

A seventh embodiment, which is the method of any one of the sixth embodiment, wherein the OCM catalyst comprises basic oxides; mixtures of basic oxides; redox elements; redox elements with basic properties; mixtures of redox elements with basic properties; mixtures of redox elements with basic properties promoted with alkali and/or alkaline earth metals; rare earth metal oxides; mixtures of rare earth metal oxides; mixtures of rare earth metal oxides promoted by alkali and/or alkaline earth metals; manganese; manganese compounds; lanthanum; lanthanum compounds; sodium; sodium compounds; cesium; cesium compounds; calcium; calcium compounds; or combinations thereof.

An eighth embodiment, which is the method of any one of the first through the seventh embodiments, wherein the OCM catalyst comprises CaO, MgO, BaO, CaO—MgO, CaO—BaO, Li/MgO, MnO₂, W₂O₃, SnO₂, MnO₂—W₂O₃, MnO₂—W₂O₃—Na₂O, MnO₂—W₂O₃—Li₂O, La₂O₃, SrO/La₂O₃, CeO₂, Ce₂O₃, La/MgO, La₂O₃—CeO₂, La₂O₃—CeO₂—Na₂O, La₂O₃—CeO₂—CaO, Na—Mn—La₂O₃/Al₂O₃, Na—Mn—O/SiO₂, Na₂WO₄—Mn/SiO₂, Na₂WO₄—Mn—O/SiO₂, or combinations thereof.

A ninth embodiment, which is the method of any one of the first through the fifth embodiments, wherein the first OCM reactor and/or the second OCM reactor exclude an OCM catalyst.

A tenth embodiment, which is the method of the ninth embodiment, wherein the first OCM reactor and/or the second OCM reactor are characterized by an OCM reactor temperature of from about 700° C. to about 1,100° C.

An eleventh embodiment, which is the method of any one of the first through the tenth embodiments, wherein at least a portion of EB is recovered from the first EB product mixture and/or the second EB product mixture.

A twelfth embodiment, which is the method of any one of the first through the eleventh embodiments, wherein at least a portion of the benzene is recovered from the first EB product mixture and/or the second EB product mixture to yield recovered benzene.

A thirteenth embodiment, which is the method of the twelfth embodiment, wherein at least a portion of the recovered benzene is recycled to the first EB reactor and/or the second EB reactor.

A fourteenth embodiment, which is the method of any one of the first through the thirteenth embodiments, wherein the benzene reacts in a liquid phase with at least a portion of the ethylene of the first EB reactant mixture and/or at least a portion of the ethylene of the second EB reactant mixture to form EB.

A fifteenth embodiment, which is the method of the fourteenth embodiment, wherein the first EB reactant mixture and/or the second EB reactant mixture are pressurized prior to introducing to the first EB reactor and/or the second EB reactor.

A sixteenth embodiment, which is the method of any one of the first through the fifteenth embodiments, wherein the first EB reactor and/or the second EB reactor are characterized by an EB reactor pressure of from about 150 psig to about 750 psig.

A seventeenth embodiment, which is the method of any one of the first through the sixteenth embodiments, wherein the first EB reactor and/or the second EB reactor comprise an acidic zeolite catalyst.

An eighteenth embodiment, which is the method of any one of the first through the thirteenth embodiments, wherein the benzene reacts in a gas phase with at least a portion of the ethylene of the first EB reactant mixture and/or at least a portion of the ethylene of the second EB reactant mixture to form EB.

A nineteenth embodiment, which is the method of any one of the first through the eighteenth embodiments, wherein an yield to EB is from about 90% to about 100%.

A twentieth embodiment, which is the method of any one of the first through the nineteenth embodiments, wherein a methane conversion is from about 5% to about 100%.

A twenty-first embodiment, which is the method of any one of the first through the twentieth embodiments, wherein equal to or greater than about 5 mol % of methane in the first OCM reactant mixture is converted overall to EB.

A twenty-second embodiment, which is the method any one of the first through the twenty-first embodiments, wherein at least a portion of the second unreacted alkanes mixture is used as a source of fuel for generating energy.

A twenty-third embodiment, which is the method of any one of the first through the twenty-second embodiments further comprising introducing additional CH₄ to the second OCM reactor.

A twenty-fourth embodiment, which is the method of any one of the first through the twenty-third embodiments, wherein the first unreacted alkanes mixture and the second unreacted alkanes mixture each comprise less than about 0.05% ethylene.

A twenty-fifth embodiment, which is the method of any one of the first through the twenty-fourth embodiments, wherein producing EB is a multi-stage process, wherein a first stage comprises steps (a) through (f), and wherein a second stage comprises steps (g) through (m).

A twenty-sixth embodiment, which is the method of the twenty-fifth embodiment, wherein the first stage and/or the second stage can be repeated as necessary to achieve a target methane conversion for the overall multi-stage process.

A twenty-seventh embodiment, which is a method for producing ethylbenzene (EB) comprising (a) introducing an oxidative coupling of methane (OCM) reactant mixture to an OCM reactor, wherein the OCM reactant mixture comprises methane (CH₄) and oxygen (O₂); (b) allowing at least a portion of the OCM reactant mixture to react via an OCM reaction to form an OCM product mixture, wherein the OCM product mixture comprises ethylene (C₂H₄), ethane (C₂H₆), water, carbon monoxide (CO), carbon dioxide (CO₂) and unreacted methane; (c) separating at least a portion of the water and optionally at least a portion of the CO and/or CO₂ from the OCM product mixture to yield an EB reactant mixture, wherein the EB reactant mixture comprises C₂H₄, C₂H₆, unreacted methane, and optionally CO and/or CO₂; (d) introducing benzene and at least a portion of the EB reactant mixture to an EB reactor, wherein the at least a portion of the EB reactant mixture is pressurized prior to introducing to the EB reactor; (e) allowing a portion of the benzene to react in a liquid phase with at least a portion of the ethylene of the EB reactant mixture to form EB; (f) recovering an EB product mixture and an unreacted alkanes mixture from the EB reactor, wherein the EB product mixture comprises EB and unreacted benzene, and wherein the unreacted alkanes mixture comprises C₂H₆ and unreacted methane, and optionally CO and/or CO₂; and (g) optionally recycling at least a portion of the unreacted alkanes mixture to the OCM reactor.

A twenty-eighth embodiment, which is the method of the twenty-seventh embodiment, wherein an yield to EB is from about 90% to about 100%.

A twenty-ninth embodiment, which is the method of any one of the twenty-seventh and the twenty-eighth embodiments, wherein a methane conversion is from about 5% to about 100%.

A thirtieth embodiment, which is the method of any one of the twenty-seventh through the twenty-ninth embodiments, wherein equal to or greater than about 5 mol % of methane in the OCM reactant mixture is converted overall to EB.

A thirty-first embodiment, which is a method for producing an ethylene derivative (ED) comprising (a) introducing an oxidative coupling of methane (OCM) reactant mixture to an OCM reactor, wherein the OCM reactant mixture comprises methane (CH₄) and oxygen (O₂); (b) allowing at least a portion of the OCM reactant mixture to react via an OCM reaction to form an OCM product mixture, wherein the OCM product mixture comprises ethylene (C₂H₄), ethane (C₂H₆), water, carbon monoxide (CO), carbon dioxide (CO₂) and unreacted methane; (c) separating at least a portion of the water and optionally at least a portion of the CO and/or CO₂ from the OCM product mixture to yield an ED reactant mixture, wherein the ED reactant mixture comprises C₂H₄, C₂H₆, unreacted methane, and optionally CO and/or CO₂; (d) introducing at least a portion of the ED reactant mixture to an ED reactor; (e) allowing at least a portion of the ethylene of the ED reactant mixture to react and form the ED; and (f) recovering an ED product mixture from the ED reactor, wherein the ED product mixture comprises the ED.

A thirty-second embodiment, which is the method of the thirty-first embodiment, wherein the ED can be selected from the group consisting of ethylbenzene, ethylene oxide, and vinyl chloride.

A thirty-third embodiment, which is a method for producing ethylbenzene (EB) comprising (a) introducing an oxidative coupling of methane (OCM) reactant mixture to an OCM reactor, wherein the OCM reactant mixture comprises methane (CH₄) and oxygen (O₂); (b) allowing at least a portion of the OCM reactant mixture to react via an OCM reaction to form an OCM product mixture, wherein the OCM product mixture comprises ethylene (C₂H₄), ethane (C₂H₆), water, carbon monoxide (CO), carbon dioxide (CO₂) and unreacted methane; (c) separating components of the OCM product mixture, wherein separating components comprises removing at least a portion of the water and optionally at least a portion of the CO and/or CO₂ from the OCM product mixture to yield an EB reactant mixture, wherein the EB reactant mixture comprises C₂H₄, C₂H₆, unreacted methane, and optionally CO and/or CO₂, and wherein separating components of the OCM product mixture excludes cryogenic distillation; (d) introducing benzene and at least a portion of the EB reactant mixture to an EB reactor; (e) allowing a portion of the benzene to react in a liquid phase with at least a portion of the ethylene of the EB reactant mixture to form EB; (f) recovering an EB product mixture and an unreacted alkanes mixture from the EB reactor, wherein the EB product mixture comprises EB and unreacted benzene, and wherein the unreacted alkanes mixture comprises C₂H₆ and unreacted methane, and optionally CO and/or CO₂; and (g) optionally recycling at least a portion of the unreacted alkanes mixture to the OCM reactor.

While embodiments of the disclosure have been shown and described, modifications thereof can be made without departing from the spirit and teachings of the invention. The embodiments and examples described herein are exemplary only, and are not intended to be limiting. Many variations and modifications of the invention disclosed herein are possible and are within the scope of the invention.

Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the detailed description of the present invention. The disclosures of all patents, patent applications, and publications cited herein are hereby incorporated by reference.

What is claimed is:

1. A method for producing ethylbenzene (EB) comprising:
(a) introducing a first oxidative coupling of methane (OCM) reactant mixture to a first OCM reactor, wherein the first OCM reactant mixture comprises methane (CH4) and oxygen (O2);
(b) allowing at least a portion of the first OCM reactant mixture to react via an OCM reaction to form a first OCM product mixture, wherein the first OCM product mixture comprises ethylene (C2H4), ethane (C2H6), water, carbon monoxide (CO), carbon dioxide (CO2) and unreacted methane;
(c) separating components of the first OCM product mixture, wherein separating components comprises removing at least a portion of the water and optionally at least a portion of the CO and/or CO2 from the first OCM product mixture to yield a first EB reactant mixture, and wherein the first EB reactant mixture comprises C2H4, C2H6, unreacted methane, and optionally CO and/or CO2;
(d) introducing benzene and at least a portion of the first EB reactant mixture to a first EB reactor;
(e) allowing a portion of the benzene to react with at least a portion of the ethylene of the first EB reactant mixture to form EB;
(f) recovering a first EB product mixture and a first unreacted alkanes mixture from the first EB reactor, wherein the first EB product mixture comprises EB and unreacted benzene, and wherein the first unreacted alkanes mixture comprises C2H6 and unreacted methane, and optionally CO and/or CO2;
(g) introducing O2 and at least a portion of the first unreacted alkanes mixture to a second OCM reactor;
(h) allowing at least a portion of the O2 and at least a portion of the first unreacted alkanes mixture to react via an OCM reaction to form a second OCM product mixture, wherein the second OCM product mixture comprises C2H4, C2H6, water, CO, CO2 and unreacted methane, and wherein an amount of unreacted methane in the second OCM product mixture is less than an amount of unreacted methane in the first OCM product mixture;
(i) separating components of the second OCM product mixture, wherein separating components comprises removing at least a portion of the water and optionally at least a portion of the CO and/or CO2 from the second OCM product mixture to yield a second EB reactant mixture, wherein the second EB reactant mixture comprises C2H4, C2H6, unreacted methane, and optionally CO and/or CO2, and wherein an amount of unreacted methane in the second EB reactant mixture is less than an amount of unreacted methane in the first EB reactant mixture;
(j) introducing at least a portion of the first EB product mixture and at least a portion of the second EB reactant mixture to a second EB reactor;
(k) allowing a portion of the benzene of the first EB product mixture to react with at least a portion of the ethylene of the second EB reactant mixture to form EB;
(l) recovering a second EB product mixture and a second unreacted alkanes mixture from the second EB reactor, wherein the second EB product mixture comprises EB and unreacted benzene, wherein an amount of unreacted benzene in the second EB product mixture is less than an amount of unreacted benzene in the first EB product mixture, wherein the second unreacted alkanes mixture comprises C2H6 and unreacted methane, and optionally CO and/or CO2, and wherein an amount of unreacted methane in the second unreacted alkanes mixture is less than an amount of unreacted methane in the first unreacted alkanes mixture; and
(m) optionally recycling at least a portion of the second unreacted alkanes to the first OCM reactor and/or the second OCM reactor.

2. The method of claim 1, wherein separating components of the first OCM product mixture and/or the second OCM product mixture excludes cryogenic distillation.

3. The method of claim 1 excluding cooling the first EB reactant mixture and/or the second EB reactant mixture.

4. The method of claim 1, wherein the first EB reactant mixture and/or the second EB reactant mixture are characterized by an EB reactant mixture temperature of from about 100° C. to about 270° C. and wherein the first EB reactor and/or the second EB reactor are characterized by an EB reactor temperature of from about 100° C. to about 270° C.

5. The method of claim 1, wherein the first OCM reactor and/or the second OCM reactor comprise an OCM catalyst selected from the group consisting of basic oxides; mixtures of basic oxides; redox elements; redox elements with basic properties; mixtures of redox elements with basic properties; mixtures of redox elements with basic properties promoted with alkali and/or alkaline earth metals; rare earth metal oxides; mixtures of rare earth metal oxides; mixtures of rare earth metal oxides promoted by alkali and/or alkaline earth metals; manganese; manganese compounds; lanthanum; lanthanum compounds; sodium; sodium compounds; cesium; cesium compounds; calcium; calcium compounds; and combinations thereof.

6. The method of claim 1, wherein the first OCM reactor and/or the second OCM reactor comprise an OCM catalyst selected from the group consisting of CaO, MgO, BaO, CaO—MgO, CaO—BaO, Li/MgO, MnO₂, W₂O₃, SnO₂, MnO₂—W₂O₃, MnO₂—W₂O₃—Na₂O, MnO₂—W₂O₃—Li₂O, La₂O₃, SrO/La₂O₃, CeO₂, Ce₂O₃, La/MgO, La₂O₃—CeO₂, La₂O₃—CeO₂—Na₂O, La₂O₃—CeO₂—CaO, Na—Mn—La₂O₃/Al₂O₃, Na—Mn—O/SiO₂, Na₂WO₄—Mn/SiO₂, Na₂WO₄—Mn—O/SiO₂, and combinations thereof.

7. The method of claim 1, wherein the first OCM reactor and/or the second OCM reactor exclude an OCM catalyst.

8. The method of claim 7, wherein the first OCM reactor and/or the second OCM reactor are characterized by an OCM reactor temperature of from about 700° C. to about 1,100° C.

9. The method of claim 1, wherein the benzene reacts in a liquid phase with at least a portion of the ethylene of the first EB reactant mixture and/or at least a portion of the ethylene of the second EB reactant mixture to form EB.

10. The method of claim 9, wherein the first EB reactant mixture and/or the second EB reactant mixture are pressurized prior to introducing to the first EB reactor and/or the second EB reactor.

11. The method of claim 9, wherein the first EB reactor and/or the second EB reactor are characterized by an EB reactor pressure of from about 150 psig to about 750 psig.

12. The method of claim 9, wherein the first EB reactor and/or the second EB reactor comprise an acidic zeolite catalyst.

13. The method of claim 1, wherein the benzene reacts in a gas phase with at least a portion of the ethylene of the first EB reactant mixture and/or at least a portion of the ethylene of the second EB reactant mixture to form EB.

14. The method of claim 1, wherein an yield to EB is from about 90% to about 100%, wherein a methane conversion is from about 5% to about 100%, and wherein equal to or greater than about 5 mol % of methane in the first OCM reactant mixture is converted overall to EB.

15. The method of claim 1, wherein at least a portion of the second unreacted alkanes mixture is used as a source of fuel for generating energy.

16. The method of claim 1 further comprising introducing additional CH₄ to the second OCM reactor.

17. The method of claim 1, wherein the first unreacted alkanes mixture and the second unreacted alkanes mixture each comprise less than about 0.05% ethylene.

18. The method of claim 1, wherein producing EB is a multi-stage process, wherein a first stage comprises steps (a) through (f), and wherein a second stage comprises steps (g) through (m), and wherein the first stage and/or the second stage can be repeated as necessary to achieve a target methane conversion for the overall multi-stage process.

19. A method for producing ethylbenzene (EB) comprising:
(a) introducing an oxidative coupling of methane (OCM) reactant mixture to an OCM reactor, wherein the OCM reactant mixture comprises methane (CH₄) and oxygen (O₂);
(b) allowing at least a portion of the OCM reactant mixture to react via an OCM reaction to form an OCM product mixture, wherein the OCM product mixture comprises ethylene (C₂H₄), ethane (C₂H₆), water, carbon monoxide (CO), carbon dioxide (CO₂) and unreacted methane;
(c) separating at least a portion of the water and optionally at least a portion of the CO and/or CO₂ from the OCM product mixture to yield an EB reactant mixture, wherein the EB reactant mixture comprises C₂H₄, C₂H₆, unreacted methane, and optionally CO and/or CO₂;
(d) introducing benzene and at least a portion of the EB reactant mixture to an EB reactor, wherein the at least a portion of the EB reactant mixture is pressurized prior to introducing to the EB reactor;
(e) allowing a portion of the benzene to react in a liquid phase with at least a portion of the ethylene of the EB reactant mixture to form EB;
(f) recovering an EB product mixture and an unreacted alkanes mixture from the EB reactor, wherein the EB product mixture comprises EB and unreacted benzene, and wherein the unreacted alkanes mixture comprises C₂H₆ and unreacted methane, and optionally CO and/or CO₂; and
(g) optionally recycling at least a portion of the unreacted alkanes mixture to the OCM reactor.

20. A method for producing ethylbenzene (EB) comprising:
(a) introducing an oxidative coupling of methane (OCM) reactant mixture to an OCM reactor, wherein the OCM reactant mixture comprises methane (CH4) and oxygen (O2);
(b) allowing at least a portion of the OCM reactant mixture to react via an OCM reaction to form an OCM product mixture, wherein the OCM product mixture comprises ethylene (C2H4), ethane (C2H6), water, carbon monoxide (CO), carbon dioxide (CO2) and unreacted methane;
(c) separating components of the OCM product mixture, wherein separating components comprises removing at least a portion of the water and optionally at least a portion of the CO and/or CO2 from the OCM product mixture to yield an EB reactant mixture, wherein the EB reactant mixture comprises C2H4, C2H6, unreacted methane, and optionally CO and/or CO2, and wherein separating components of the OCM product mixture excludes cryogenic distillation;
(d) introducing benzene and at least a portion of the EB reactant mixture to an EB reactor;
(e) allowing a portion of the benzene to react in a liquid phase with at least a portion of the ethylene of the EB reactant mixture to form EB;
(f) recovering an EB product mixture and an unreacted alkanes mixture from the EB reactor, wherein the EB product mixture comprises EB and unreacted benzene, and wherein the unreacted alkanes mixture comprises C2H6 and unreacted methane, and optionally CO and/or CO2; and
(g) optionally recycling at least a portion of the unreacted alkanes mixture to the OCM reactor.
